(19) United States
(12) Patent Application Publication
(10) Pub. No.: US 2010/0217388 A1
(43) Pub. Date: Aug. 26, 2010

(54) SYSTEM AND METHOD FOR RESHAPING SOFT TISSUE

(75) Inventors: Adi Cohen, Hod Hasharon (IL); Benny Dilmoney, Givat Shmuel (IL)

Correspondence Address: THE NATH LAW GROUP, 112 South West Street, Alexandria, VA, 22314, US

(73) Assignee: ORBIX MEDICAL LTD, Kiryat Shmona (IL)

(21) Appl. No.: 12/738,085

(22) PCT Filed: Oct. 22, 2008
(86) PCT No.: PCT/IL08/01360

§ 371 Date: Apr. 15, 2010

Related U.S. Application Data
(60) Provisional application No. 60/960,831, filed on Oct. 16, 2007.

(51) Int. Cl. A61F 2/12; A61B 17/00

(52) U.S. Cl. 623/8; 606/151

(57) ABSTRACT

Provided is a system and method for soft tissue shaping (such as breast, buttocks tissues, arms and neck lifting and shaping/reshaping), including a cradling member for cradling soft tissue and an anchoring system for fixing the cradling member to posture tissue in at least one location. The cradling member has a top side and bottom side, wherein at least one of the top side or bottom side may be fitted with at least one inflatable compartment.

### FIELD OF THE INVENTION

This invention relates to the field of soft tissue support, lift and shape correction. The system according to the present invention may be applied for the purpose of breast shape correction, by altering the shape of an individual's breast or other soft tissues such as buttocks, arm or neck tissues.

### BACKGROUND OF THE INVENTION

Over the years, factors such as pregnancy, nursing, and the force of gravity take their toll on a woman's breasts. This situation is known as ptosis and is defined as a to situation at which the nipple-areola complex projection is lower then the infra-mammary fold, i.e. the nipple is below the level of the lower breast crease.

As the skin loses its elasticity, the breasts often lose their shape and firmness and begin to sag, obtaining a teardrop like shape rather then a cone-like shape. Breast lifting and reshaping, also referred to by the alternative names mastopexy and mammoplasty, is a procedure used for reshaping saggy and loose breasts, elevating the nipple and areola to a higher level and thus affording the breast its former shape and firmness which can result in a revitalized body image that can bolster a woman's self-esteem.

Many women use a push-up bra to support their breasts. This however is at times uncomfortable and may be restricting as far as the selection of clothing.

Mastopexy procedure according to prior art techniques involves reducing ptosis (sagging of the breast caused by stretched skin, in many cases due to a great loss of breast tissue). During a breast lift, long incisions are made along the natural creases in the breast and around the dark skin surrounding the nipple (areola), a keyhole-shaped incision above the areola is also made to define the new location for the nipple. Excess skin is removed from the lower section of the breast and the areola, nipple, and underlying breast tissue is repositioned up to a higher position. The nipple is moved and incisions are closed with sutures.

Several methods for performing a mastopexy are known, and the technique opted for depends mainly on the amount of breast and fat tissue, the amount of skin to be removed, symmetry in volume of breasts and size of areolas, and choice and taste of patient. Since the procedure involves surgery, it may be coupled with breast augmentation and with resizing or repositioning of the areola to a more aesthetically pleasing position and the shape of the mound may be improved by placement of breast implants. Occasionally, only a one-sided breast lift is required, when the other breast is in a reasonable position on the chest and does not require a breast lift.

U.S. Pat. No. 5,676,161 to Breiner discloses a mastopexy procedure according to which an anchor-shaped incision is made, having a bottom line along the infra-mammary crease, using a circular cutter to form the top portion of the incision and an incision around the areola to reduce the diameter thereof. After removing excess skin inside the incision, and breast tissue in the case of mammaplasty, shifting the areola, nipple and underlying breast tissue upward to position the areola/nipple complex within the circular top portion, pulling the flaps of skin formed to the sides of the incision down and around the areola and underneath same, and then suturing adjacent skin edges to complete the lifting and reshaping.

U.S. Pat. No. 5,584,884 to Pignataro discloses a mammary prosthesis comprising a wedge shaped sheet of flexible biocompatible material having reinforced upper and lower attachment portions for attachment to bone of a patient by bone anchors, with the lower attachment portion being anchored to one or more ribs. The lower attachment portion includes a support member less flexible than the sheet material having suture receiving openings for receiving bone anchor sutures.

French publication No. 2746298 to Bellity discloses a support prosthesis having a cover to enclose the organ an at least one section for fixing of the prosthesis in the body. The cover can have a form corresponding to the normal anatomy of a breast.

French publication No. 2682284A1 to Dessapt discloses a mammary prosthesis which can be incorporated under the skin of a patient, and designed to shape and support the breast of the patient, comprising a collar in the form of a flexible blade, at least in part open worked, made of a biocompatible material. The prosthesis is characterized in that the collar is provided with two asymmetric branches designed to close on each other and to be fixed at least partly onto each other, in order to form a framework which can at least partially enclose and envelope the breast and have a bulged shape similar to that of a natural breast, the surface area of the open worked part of the collar which has no material being larger than the surface area of the open worked part of the collar occupied by the material.

European publication No. 0230672A2 to Campbell discloses a method for implantation of a mammary prosthesis involving interposing a bather layer of sort biological tissue at the interface of the prosthesis and the surrounding host tissue. This bather layer reduces the formation of fibrous connective tissue capsules and subsequent hardening of the scar tissue that may occur following implantation of mammary prosthesis.

These procedures are typically performed under general anesthesia, though at times local anesthesia is sufficient, and they may last several hours, depending on the extent of the surgery.

As with any surgery, there is always a possibility of complications such as a reaction to the anesthesia, bleeding and infection (which may cause scars to widen). Mastopexy does leave noticeable, permanent scars, although are so planed as to be concealed by a woman's bra or bathing suit. One may expect that after about one year the scars will hardly be noticeable. As far as aesthetics, there may also be some unsatisfactory results, as the final appearance may not always meet the patient's expectations. Evermore, a breast lift performed according to conventional techniques won't keep firm forever, the effects of gravity, pregnancy, aging, and weight fluctuations will eventually take their toll again.

Other areas of the human body such as arms, neck, buttocks and other areas with soft tissues are affected by factors such as age, weight lose and gravity force which take their toll on these tissues. The drooping of skin is a result of stretching of the natural anchoring system and loss of supporting fat.

An arm lift, also known as brachioplasty, is a surgical procedure to remove loose skin and excess fat deposits along the upper arm. Brachioplasty is performed to remove excess tissue and reduce the circumference of the upper arm. Such surgical procedure bears a possibility of complications such as a reaction to the anesthesia, bleeding and infection (which may cause scars to widen).

A neck lift, or platysmaplasty and even submental platysmaplasty, is a surgery designed to reduce the loose look of sagging skin, or a “turkey waddle”, in the neck area and under the jaw line. In such a procedure sections of skin are trimmed and lifted into place and sutured or fixed with biological tissue glue. With the platysmaplasty, a section of fat and muscle is removed and the ends are sutured to bring them together at the mid-anterior (front) section of the neck. The skin can be brought together under or behind the ear to further firm up appearance of the neck. According to some surgical procedures, suture, mesh or even AlloDerm suspension as a sort of hammock are used to keep the neck tight and waddle-free. Brachioplasty and platysmaplasty often leave noticeable, permanent scars and often some unsatisfactory results, as the final appearance may not always meet the patient's expectations.

It is an object of the present invention to provide a novel system and a method for soft tissue such as breast, buttocks tissues, arms and neck lifting and shaping/reshaping, wherein the above drawbacks are significantly reduced or overcome. The system according to the invention and the method for carrying it out are minimally invasive and may be considered as non-surgical, i.e. they do not involve incisions (but rather two or four stab incisions) and removal of excess skin, nor do they require stitches. However, the system may also be used in conjunction with breast enlargement (breast augmentation with mastopexy) or breast size reduction, which are surgical procedures.

### SUMMARY OF THE INVENTION

The present invention is directed towards a system and method for re-shaping and supporting soft tissue of a patient.

A significant advantage of the present invention, apart from the fact that it is a minimally invasive procedure, is that the aesthetic results and appearance may be modified to match with customer's expectations during, or any time after the procedure, i.e. corrections may be easily effected after a while if ptosis reoccurs. Attaching the soft tissue to a posture tissue will prevent re-sagging and will accomplish a long-lasting aesthetic result.

A soft tissue shaping system according to one aspect of the present invention comprises a cradling member for cradling the soft tissue, and an anchoring system for fixing the cradling member to posture tissue in at least one location, the cradling member having at least a top side and a bottom side, wherein at least one of the at least the top side or the bottom side is fitted with at least one inflatable compartment.

A soft tissue shaping system according to another aspect of the present invention comprises a cradling member for cradling the soft tissue, and an anchoring system for fixing the cradling member to posture tissue in at least one location above the center of gravity of the soft tissue.

A method for shaping a soft tissue of a patient, according to another aspect of the present invention, comprises the following steps:
a) providing a cradling member for cradling the soft tissue and an anchoring system for fixing the cradling member to posture tissue in at least one location, the cradling member having at least a top side and a bottom side;
b) forming at least one stab-incisions at part of the soft tissue;
c) forming a transverse passage through the soft tissue, extending between said stab-incisions;
d) introducing the cradling member through the transverse passage;
e) fixating the anchoring system through the stab incisions;

According to one embodiment, the cradling member further comprises at least one inflatable compartment. According to this embodiment the inflatable compartments may be filled either before step (d) or thereafter. According to another embodiment, following step (e) at least one of the at least one inflatable compartment may be inflated. According to still an embodiment of the present invention inflation may be carried out while in an upright position of the patient.

According to another embodiment, the cradling member may be integrated with the anchoring system and step (e) may be carried out by directly fixating the anchoring system to the posture tissue.

Any one or more of the following features and characteristics may be implemented in the system and method according to the present invention:
each of the top side and the bottom side may be fitted with at least one compartment adapted to be filled/inflated;
at least one of the top side and the bottom may be provided with several inflatable compartments, each being adapted to inflate and reshape the soft tissue;
the at least one inflation compartment may be provided with an inflation/deflation valve;
the at least one inflatable compartment may be filled with a biocompatible material such as gas, a saline solution, a silicone gel, a hydro gel or the like;
the soft tissue may be a breast tissue, arm tissue, neck tissue or buttocks tissue or other soft tissue;
the soft tissue may be a breast tissue and the anchoring system may comprise one or more anchors adapted to be fixed to a posture tissue with one or more suspending members suspended from the one or more anchors and may extend through the breast for supporting the cradling member;
the soft tissue may be a breast tissue and the posture tissue is located above a desired nipple level;
the posture tissue may be located above the center of gravity of the soft tissue;
the soft tissue may be buttocks tissue and the anchoring system may comprise one or more anchors adapted to be fixed to a posture tissue, with one or more suspending members suspended from the one or more anchors and extending through the tissue for supporting the cradling member;
the soft tissue may be arm tissue and the anchoring system may comprise one or more anchors adapted to be fixed to a posture tissue, with one or more suspending members suspended from the one or more anchors for supporting the cradling member;
the soft tissue may be neck tissue and the anchoring system may comprise one or more anchors adapted to be fixed to a posture tissue, with one or more suspending members suspended from the one or more anchors for supporting the cradling member;
the anchor may be a bolt fixture or a threaded fixture fixed to a rib or a collar bone, in case of the reshaping of the buttocks tissue the posture tissue may be a Pelvic girdle bone, in case of the reshaping of the neck tissue he posture tissue may be a mandibular bone, or in case of the reshaping of the arm tissue the posture tissue may be a humerus bone.
said soft tissue may be breast tissue and the cradling member may be attached to a posture tissue along an inframammary fold;
said soft tissue may be breast tissue and the cradling member may be attached to a posture tissue along an inframammary fold and may further comprise and anchoring system positioned above the desired nipple level with one or more suspending members suspended from the one or more anchors wherein the anchor may be a bolt fixture or a threaded fixture fixed to a rib or a collar bone;
the anchoring system may comprise one or more support members integral with or articulated to the cradling member;
the support members may be fitted to either or both the top side and the bottom side of the cradling member;
the support members may be in the form of tabs laterally projecting from a longitudinal edge of the cradling member fitted for bearing against posture tissue;
at least a portion of the cradling member may be made of a bio-compatible material, mesh like material, silicon sheet, silicon sheet comprising embedded mesh-like material, reinforced silicon material, silicon sheet with mesh-like material with non-homogeneous reinforcing qualities etc; and
at least a portion of the cradling member may be a mesh-like portion.

### BRIEF DESCRIPTION OF THE DRAWINGS

In order to understand the invention and to see how it may be carried out in practice, embodiments will now be described, by way of non-limiting examples only, with reference to the accompanying drawings, in which:

FIGS. 1A-1C illustrate a front view, isometric view and a transverse cross-sectional view, respectively, of a soft tissue shaping system comprising inflatable compartments on the top side of the cradling member according to one embodiment of the present invention;

FIGS. 2A-2C illustrate a front view, isometric view and a transverse cross-sectional view, respectively, of a soft tissue shaping system comprising inflatable compartments on the bottom side of the cradling member according to one embodiment of the present invention;

FIGS. 3A-3C illustrate a front view, isometric view and a transverse cross sectional view, respectively, of a soft tissue shaping system comprising inflatable compartments on the top side and the bottom side of the cradling member according to one embodiment of the present invention;

FIGS. 4A-4B schematically illustrate a side, cross-sectional view of a top portion of female torso, superimposing how the soft tissue shaping system supports the breast before and after the inflation, respectively, of the inflatable compartment on the bottom side of the cradling member;

FIGS. 5A-5B schematically illustrate a side, cross-sectional view of a top portion of the female torso, superimposing how the soft tissue shaping system supports the breast before and after the inflation, respectively, of the inflatable compartment on the top side of the cradling member;

FIGS. 6A-6B schematically illustrate a side, cross-sectional view of a top portion of the female torso, superimposing how the soft tissue shaping system supports the breast before and after the inflation of the inflatable compartments on both the top side and the bottom side of the cradling member, respectively;

FIG. 7 schematically illustrates a top portion of female torso, superimposing how shaping system according to any one of the embodiments of FIGS. 1A-3C supports the breast tissue;

FIG. 8 schematically illustrates how the present invention is used for reshaping an individual's buttock tissue according to one embodiment of the present invention;

FIG. 9 illustrates a cradling member according to one embodiment of the present invention;

FIG. 10 schematically illustrates how the present invention is used for reshaping an individual's arm tissue according to an embodiment of the present invention; and

FIG. 11 schematically illustrates how the present invention is used for reshaping an individual's neck tissue according to another embodiment of the present invention.

### DETAILED DESCRIPTION OF EMBODIMENTS

The system of the present invention generally comprises a cradling member and an anchoring system. The anchoring system secures the cradling member in a desired position and is fixed to a posture tissue in at least one location.

A posture tissue according to the present invention may be a bone (i.e. ribs or collar bone in the case of breast tissue, pelvic bone in a case of buttocks tissue, humerus bone in case of arm tissue and mandibular bone in case of neck tissue), though it may also be muscle tissue or fascia.

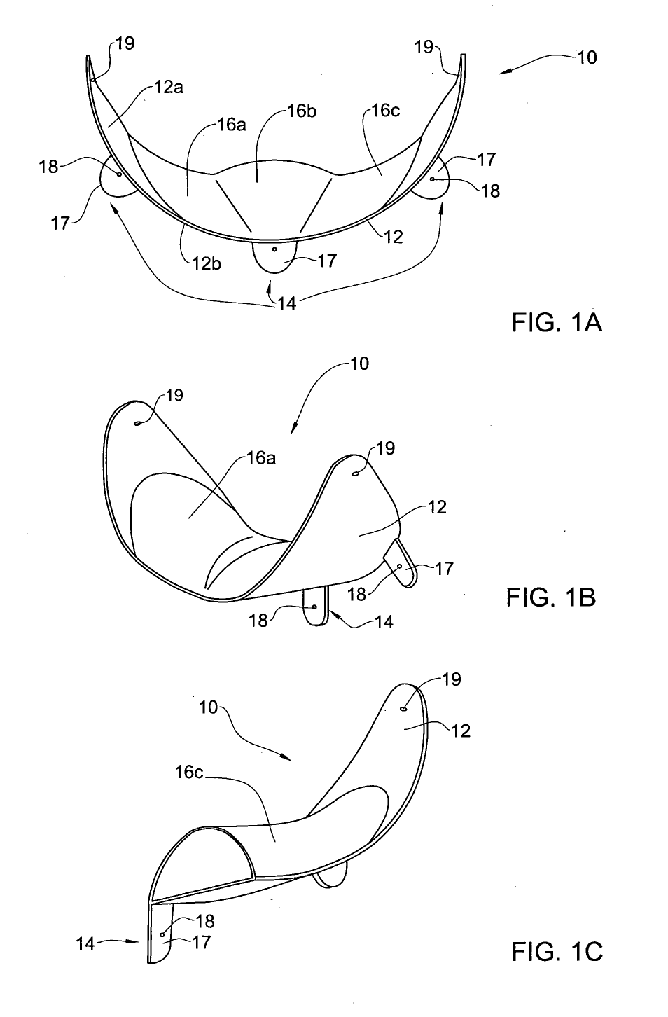

Referring first to FIG. 1A, a soft tissue shaping system 10 according to one example of the present invention comprises a cradling member 12 for cradling a breast tissue and an anchoring system 14 for fixing the cradling member 12 to posture tissue, i.e. ribs or collar bone, at three locations.

The cradling member 12 has a top side 12a and a bottom side 12b and further comprises according to this example three inflatable compartments 16a, 16b, 16c on the top side 12a thereof.

The cradling member 12 and the anchoring system 14 may be formed as an integrated homogeneous unit formed from a flexible biocompatible material. According to the exemplified system, the cradling member 12 and the anchoring system 14 are formed seamlessly, for example by molding, in order to eliminate seams, sharp edges, mold flashing and the like. According to another example the anchoring system 14 may be non-integrated with the cradling member 12 and articulated thereto by other arrangements.

The cradling member 12 may be typically formed to appear rounded, hemispherical or anatomically shaped. In the presently contemplated example, the cradling member 12 is substantially hemispherical and the anchoring system 14 comprises three flat anchoring tabs laterally projecting from a rear longitudinal edge of the cradling member 12 which in use is adapted to lie along the inframammary fold. However, the tabs of the anchoring system 14 may be provided on top face 12a and/or bottom face 12b of the cradling member 12. These anchoring tabs 17 secure the cradling member 12 to the individual's posture tissue as discussed hereinabove. The anchoring tabs 17 are provided with apertures 18 which may be formed during the manufacturing process or simply by perforation during the process of anchoring the tabs 17 to the posture tissue by appropriate anchors such as self tapping screws, stitches and the like. According to another example, the cradling member may be provided with additional apertures 19 adapted to receive suspending members therethrough (best seen in FIG. 1B). The suspending members may be in a form of a strap made from a biocompatible material and suspended from an anchor adapted to be fixed to a posture tissue.

It is desirable that the cradling member 12 and the anchoring system 14 be as thin as possible without compromising its effectiveness to support the soft tissue such as the breast or the buttocks tissues. A cradling member having a thickness ranging from 0.5 millimeters through 3 millimeters is contemplated as being sufficient to support tissue and is believed to function satisfactorily in a majority of cases. With respect to the anchoring system, it is designed to have thickness determined on an individual case basis. The cradling member material may be entirely or partially reinforced with reinforcing ribs or any suitable reinforcing material such as reinforced silicon mesh material.

The cradling member may be made from rigid though pliable material such that it may be deformed to any desired shape and retain its deformed shape for complying with the individual's anatomy and required resultant shape. For example, the cradling member may be made of or reinforced by a memory shaped metal, plastic material deformed under heat, a biocompatible material (e.g. in a form of a mesh-like material), silicon sheet, silicon sheet embedded with a mesh-like material, reinforced silicon material, silicon sheet with mesh-like material with non-homogeneous reinforcing qualities i.e., having different mechanical properties (shown in FIG. 9).

As illustrated in FIG. 9, the cradling member is a silicon sheet comprising embedded mesh like material with non-homogeneous reinforcing qualities. The portion of the cradling member designated A has different mechanical properties from the area designated B. Area B is adapted to sustain gravitational forces on the cradling member and maintain its shape. The anchoring system according to an embodiment of this invention may also be reinforced to provide a better support.

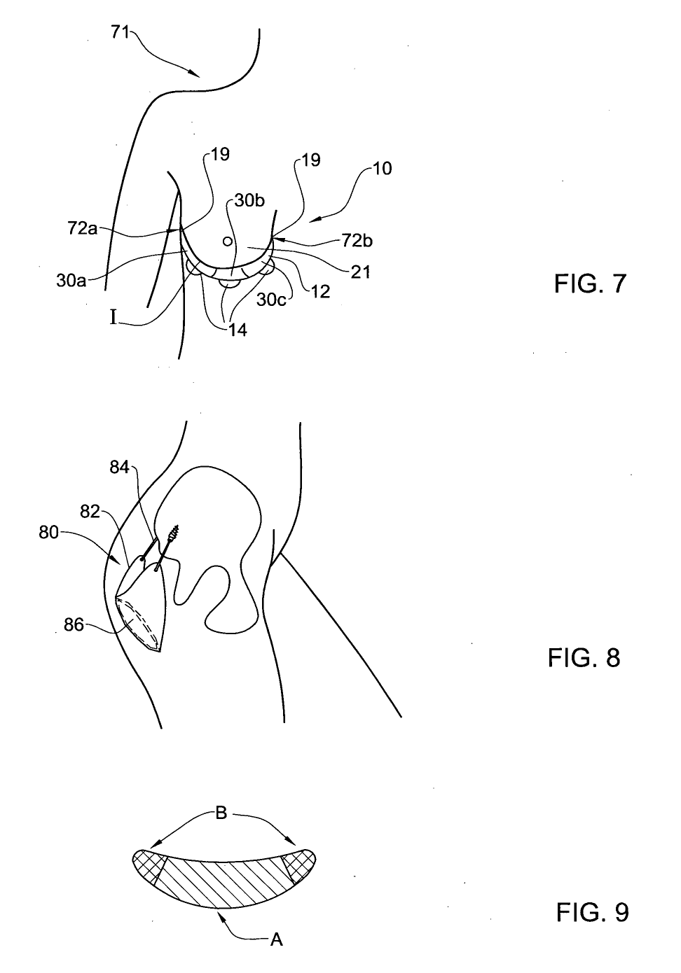

The soft tissue shaping system 10 may be utilized to support any desired soft tissue. According to one example shown in FIG. 7 the soft tissue 21 is breast tissue and the system 10 is positioned such that the cradling member 12 cradles the breast 21 from below in a manner that the cradling member 12 is aligned and secured along the inframammary fold I.

The inflatable chambers 16a-16c are integrated with the cradling member 12 in the desired position and are formed of a flexible, elastically deformable material capable of inflation/deflation without the deterioration of elasticity thereof. During installation (i.e. insertion and anchoring), or before or after insertion of the system, the inflatable chambers may be inflated by introduction of suitable material. Typical inflation materials may include any fluids such as gases, biocompatible solutions, silicone gel, saline solution, hydro gel or the like The inflation materials may be chosen for their density, viscosity, biocompatibility, antimicrobial nature, stability over time and the like.

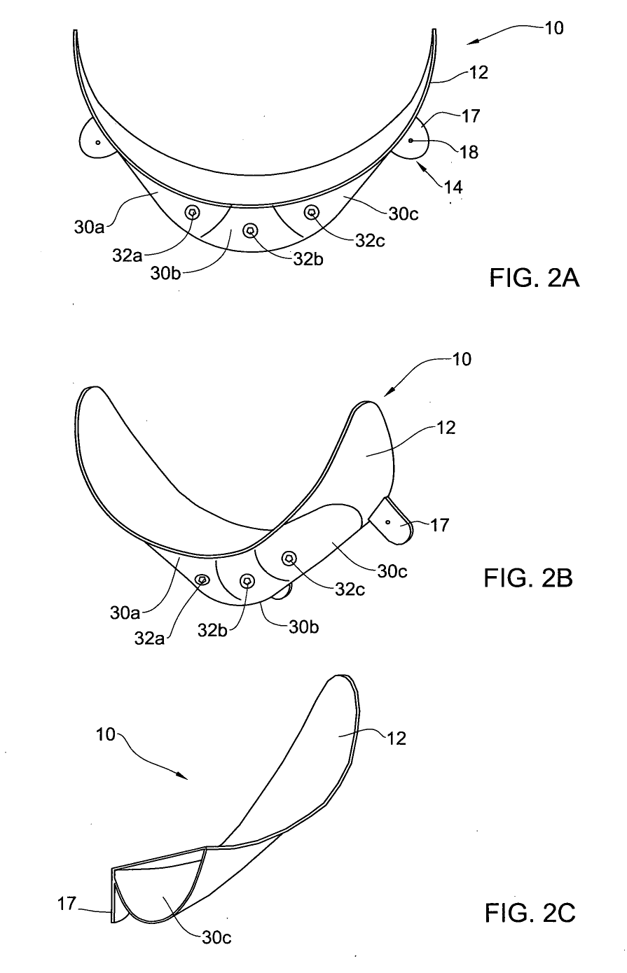

As can be further seen in the embodiment exemplified in FIGS. 2A-2B each of the inflatable compartments 30a, 30b, 30c is fitted with an inflation/deflation valve 32a, 32b, 32c respectively, for facilitating inflation or deflation upon individual demand. Such valves may be in the form of a self sealing resilient membrane wherein inflation fluid is introduced or withdrawn by a use of a syringe needle piercing through the valve.

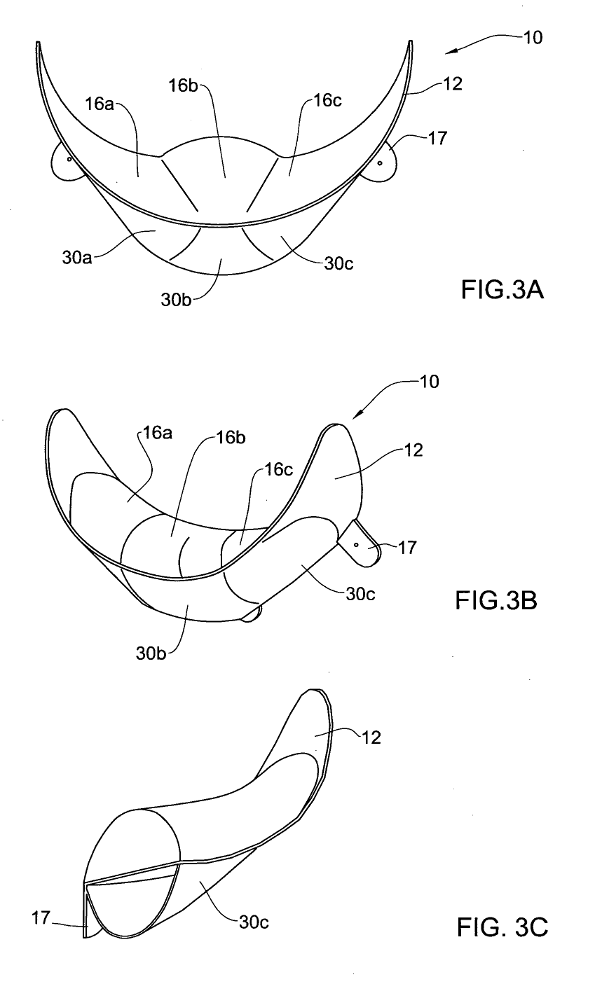

In order to obtain a desired shape of the soft tissue the inflatable chambers 16a-16c may be affixed to the top side 12a of the cradling member 12 (seen in FIGS. 1A-1C), the bottom side 12b thereof (seen in FIGS. 2A-2C) or both (seen in FIGS. 3A-3C). The cradling member 12 may be provided with a single chamber only or may comprise several inflatable chambers 16a-16c as described above, integrally fitted either continuously or at desired locations along the cradling member 12. Furthermore, the inflatable chambers may each assume a different shape to impart the manipulated breast or any other soft tissue any desired shape and size.

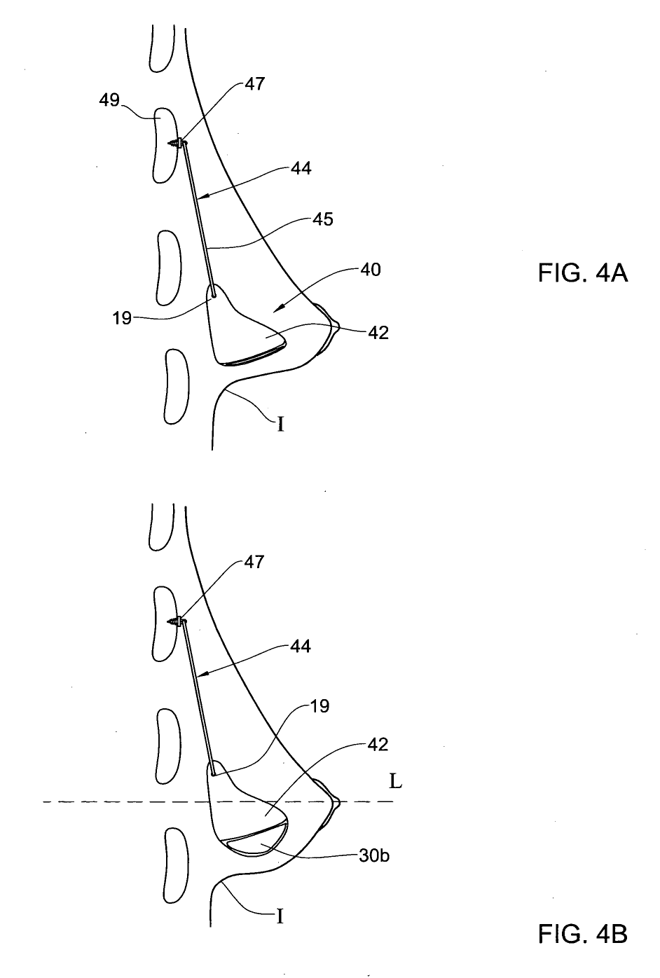

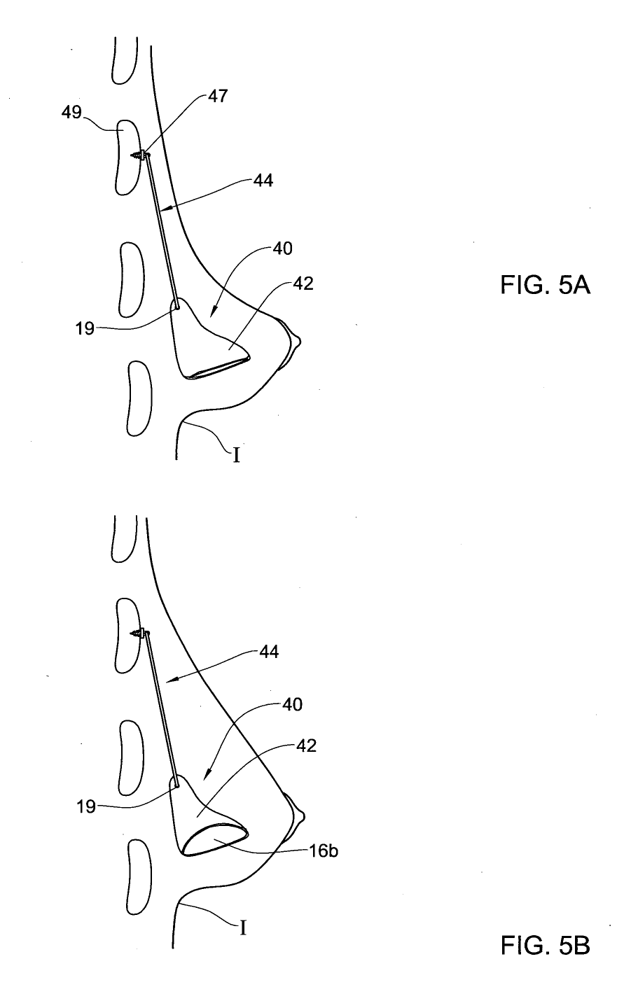

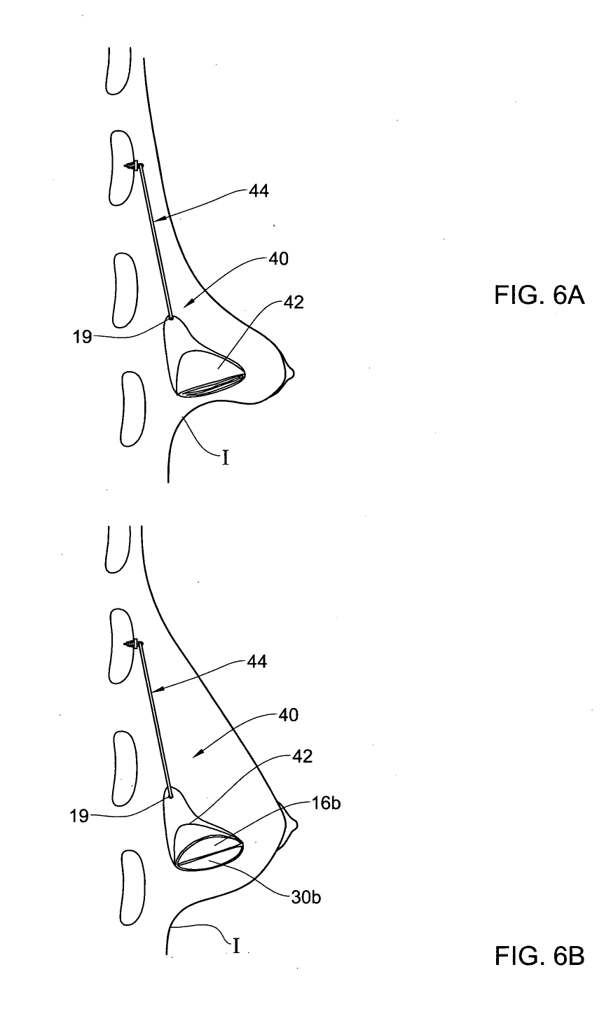

According to additional example illustrated in FIGS. 4A-6B, a soft tissue support system 40 comprises a cradling member 42 and an anchoring system 44. As already mentioned above, the cradling member 42 may be typically formed to appear rounded, hemispherical or otherwise anatomically shaped.

In the presently contemplated example, the cradling member 42 is substantially hemispherical and the anchoring system 44 comprises two suspension members 45 in the form of cords (e.g. at least partially formed from a tendon-like wire or a mash, made of polyethylene, polyester, polyblend, organic material such as tendons, or synthetic material e.g. silicone, Gortex™, etc.) and corresponding anchors 47 fixed to a posture tissue (rib 49), above a desired nipple level L (FIG. 4B). Adjusting the length (shortening/lengthening) entails corresponding lifting or lowering of the breast. The cradling member may be fully or partially reinforced.

For each breast, anchors may be fixed to one or more posture, depending on the physiology/anatomy of the patient, the shape of the breast prior to shaping, and the desired shaping result. An anchor may be a bolt fixture or a threaded fixture, typically but not restricted thereto, a self tapping screw for screw-fixation into a bone, a suspending hook for bearing from a bone, i.e. clinging from the bone, or a clasp formed with hooks for grasping soft tissue (muscle). Alternatively, where the posture tissue is a muscle or fascia, the suspension member may be fixed to soft tissue thereto by stitching or soft tissue anchoring device. According to still an alternative, the suspension member may be attached to a bone by tying or yarning it through a bore formed through the bone.

As can be seen in the embodiment of FIG. 7, during implantation, at a first step, after local anesthesia of the area surrounding the soft tissue 21 of a patient 71 (likely only local anesthesia is required), two stab incisions 72a and 72b are formed around the soft tissue 21 followed by a step of transverse passage formation between the incisions 72a and 72b to insert the system 10 through the passage. Anchoring system 14 is then attached to the patient's posture tissue and thus the cradling member 12 is fixated to the posture tissue. At least one of the inflatable compartments 32a, 32b, 32c is then inflated, this step is preferably carried out while the patient 71 is in an upright position, such that actual indication is available regarding the breast's 21 new form and position, and even more so, the patient may take part in deciding to what extent to lift the breasts.

The inflatable compartments as mentioned above may be filled prior to, during or at any time after the implantation of the system and adjustments of both the suspension members and extent of inflation of the inflatable compartments.

Shortly after the implantation procedure, the patient may be released, with complete healing expected in a matter of days, essentially not leaving any noticeable scars.

The embodiment of FIG. 8 illustrates how the system and the method according to the present invention may be utilized for reshaping buttocks tissue. The system 80 and method is substantially similar to the disclosed hereinabove system and method described with reference to a breast tissue. According to an example of the invention, the system may be devoid the inflatable compartments. Owing to the nature and anatomy of the buttocks tissue and its location, its is appreciated that modifications are required such as reinforcement of the cradling member 82 and the anchoring system 84 and the provision of multiple anchoring sites for securing the system to a pelvic bone. Also, it is appreciated that the one or more inflatable compartments 86 are substantially larger that those concerned with breast tissue augmentation.

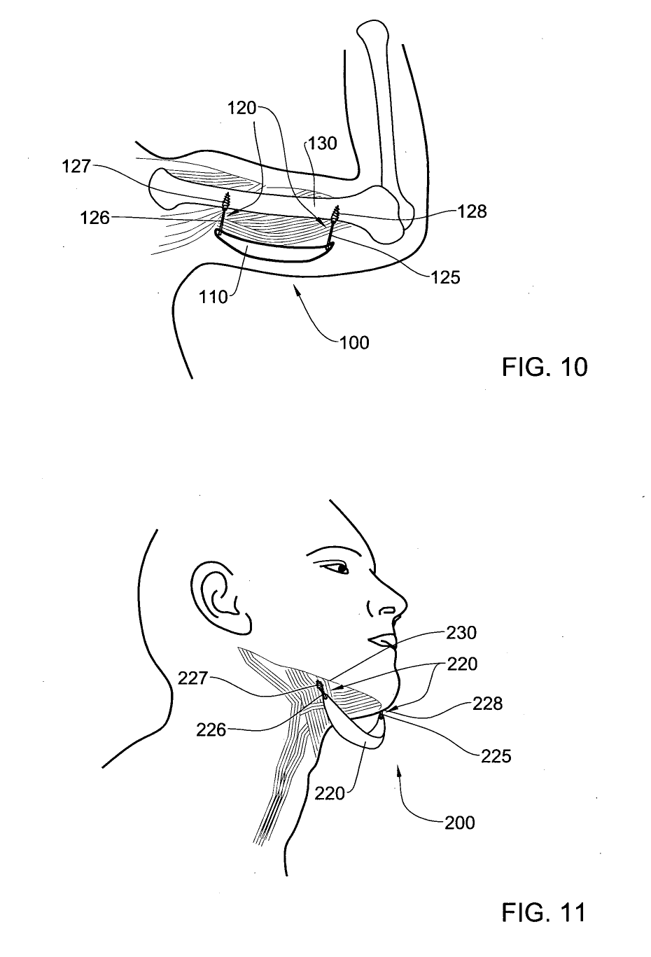

FIG. 10 is schematic illustration of yet another implementation of the invention showing the system as described hereinabove for reshaping and lifting arm tissue. According to this example, the system, generally designated 100, has a cradling member 110 for cradling loose skin and excess fat tissue and an anchoring system 120 for fixing the cradling member 110 to posture tissue, i.e. a humerus bone.

As shown in FIG. 10 the cradling member 110 is suspended from the posture tissue 130. The anchoring system comprises two suspension members 125 and 126 in the form of cords (e.g. at least partially formed from a tendon like wire or a mesh, made of polyethylene, polyester, polyblend, organic material such as tendons, or synthetic materials such as silicone etc.) and corresponding anchors 127 and 128 fixed to the posture tissue 130. The cords 125 and 126 may be adjusted in length and position to entail corresponding lifting or lowering of the soft tissue.

As mentioned hereinabove, the cradling member 110 may be formed to undertake a desired anatomical shape and may be made of various materials and designed substantially as described above.

FIG. 11 is schematic illustration of yet an implementation of the invention showing the system as described hereinabove for reshaping and lifting neck tissue. According to this example, the system, generally designated 200, has a cradling member 210 for cradling loose skin and excess fat tissue and an anchoring system 220 for fixing the cradling member 210 to posture tissue, i.e. mandibular bone.

As shown in FIG. 11 the cradling member 210 is suspended from the posture tissue 230. The anchoring system comprises two suspension members 225 and 226 in the form of cords (e.g. at least partially formed from a tendon like wire or a mesh, made of polyethylene, polyester, polyblend, organic material such as tendons, or synthetic materials such as silicone etc.) and corresponding anchors 227 and 228 fixed to the posture tissue 230. The cords 225 and 226 may be adjusted in length and position to entail corresponding lifting or lowering of the soft tissue.

As already mentioned hereinabove, the cradling member 210 may formed to undertake a desired anatomical shape and may be made of various materials and designed substantially as described above.

Whilst some embodiments have been described and illustrated with reference to some drawings, it will be appreciated that many changes may be made therein without departing from the general spirit and scope of the invention, mutatis, mutandis.

1.-41. (canceled)

42. A soft tissue shaping system comprising a cradling member for cradling the soft tissue, and an anchoring system for fixing the cradling member to posture soft tissue in at least one location, the cradling member having at least a top side and a bottom side, wherein at least one of the at least the top side and the bottom side is fitted with at least one inflatable compartment.

43. The system according to claim 42, wherein each of the top side and the bottom side is fitted with at least one inflatable compartment.

44. The system according to claim 42, wherein at least one of the top side and the bottom is provided with several inflatable compartments, each being adapted to inflate and reshape the soft tissue.

45. The system according to claim 42, wherein the at least one inflation compartment is provided with an inflation/deflation valve.

46. The system according to claim 42, wherein the at least one inflatable compartment is filled with a biocompatible material such as gas, a saline solution, a silicone gel, a hydro gel or the like.

47. The system according to claim 42, wherein the soft tissue is a breast tissue, buttocks tissue, arm tissue or neck tissue.

48. The system according to claim 42, wherein the soft tissue is neck tissue, buttocks tissue or arm tissue and the anchoring system is fixed to a posture tissue located above the center of gravity of the respective soft tissue.

49. The system according to claim 42, wherein the anchoring system includes one or more support members integral with or articulated to the cradling member.

50. The system according to claim 49, wherein the support members are in the form of tabs laterally projecting from a longitudinal edge of the cradling member fitted for bearing against posture tissue.

51. A method for shaping a soft tissue of a patient, the method comprising the following steps:
providing a cradling member for cradling the soft tissue and an anchoring system for fixing the cradling member to posture tissue in at least one location, the cradling member having at least a top side and a bottom side;
forming at least one stab-incision at a lower part of the soft tissue;
forming a transverse passage through the soft tissue;
introducing the cradling member through the transverse passage;
fixating the anchoring system through the stab incisions.

52. The method according to claim 51, wherein the cradling member further includes at least one inflatable compartment;

53. The method according to claim 51, wherein the cradling member is integrated with the anchoring system and wherein step (e) is carried out by directly fixating the anchoring system to the posture tissue.

54. The method according to claim 52, wherein at least one of the at least one inflatable compartments is inflated prior to step (d).

55. The method according to claim 52, wherein following step (e) at least one of the at least one inflatable compartment is inflated.

56. The method according to claim 55, wherein inflation is carried out while in an upright position of the patient.

57. The method according to claim 51, wherein the soft tissue is breast tissue, neck tissue buttocks tissue or arm tissue and the anchoring system includes one or more anchors adapted to be fixed to a posture tissue with one or more suspending members suspended from the one or more anchors for supporting the cradling member.

58. The method according to claim 51, wherein the posture tissue is above a desired nipple level or a center of gravity of the soft tissue.
